(19) United States
(12) Plant Patent Application Publication
(10) Pub. No.: US 2014/0223626 P1
(43) Pub. Date: Aug. 7, 2014

(54) Dendrobium orchid plant 'SPCDW1005'

(50) ;

(71) Applicant: Thumrong Suphachadiwong, Chonburi (TH)

(72) Inventor: Thumrong Suphachadiwong, Chonburi (TH)

(73) Assignee: Suphachatwong Innovation Co, Chonburi (TH)

(21) Appl. No.: 13/999,298

(22) Filed: Feb. 6, 2014

(30) Foreign Application Priority Data
Feb. 7, 2013 (QZ) A201300222

(51) Int. Cl. A01H 5/00

(52) U.S. Cl. PLT/311

(57) ABSTRACT

A new and distinct Dendrobium cultivar named ‘SPCDW1005’ is disclosed, characterized by distinctive petal and sepal shape, unique petal and sepal color and predominantly purple lip color. The new variety is a Dendrobium, typically produced as an indoor ornamental plant.

### LATIN NAME OF THE GENUS AND SPECIES

Dendrobium hybrid.

### VARIETY DENOMINATION

‘SPCDW1005’

### BACKGROUND OF THE INVENTION

This application relates to a new cultivar of Dendrobium hybrid. The new variety is the product of a planned breeding program. The new variety originated as a seedling from the crossing of the unpatented, proprietary seed parent Dendrobium ‘BJ’ with the unpatented, proprietary pollen parent referred to as Dendrobium ‘181N’. The crossing was made by the inventor in his research greenhouse in Chonburi, Thailand in December of 1999.

The new variety was first selected by the inventor, Thumrong Suphachadiwong, a citizen of Thailand, in February of 2003 in a research greenhouse belonging to the inventor in Chonburi, Thailand. After identifying the new variety as a potentially interesting selection, the inventor continued confidential testing and propagation of ‘SPCDW1005’, assessing stability of the unique characteristics of this variety.

Asexual reproduction of the new cultivar ‘SPCDW1005’ was first performed at a commercial laboratory belonging to the inventor in Chonburi, Thailand by tissue culture in September of 2005. Access to all plants was restricted, as plants were kept in a greenhouse not open to the public, and tissue culture plantlets were in a laboratory belonging to the inventor, and not accessible to the public. Through subsequent propagation, multiple generations have been reproduced, which have shown that the unique features of this cultivar are stable and reproduced true to type.

### SUMMARY OF THE INVENTION

The cultivar ‘SPCDW1005’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘SPCDW1005.’ These characteristics in combination distinguish ‘SPCDW1005’ as a new and distinct Dendrobium cultivar:

1. Distinctive petal and sepal shape.

2. Green with purple sepal and petal color.

3. Predominantly purple lip color.

### PARENT COMPARISON

Plants of the new cultivar ‘SPCDW1005’ are similar to the seed parent, Dendrobium ‘BJ’ in most horticultural characteristics. However the new variety, ‘SPCDW1005’ differs from ‘BJ’ in the following characteristics:

1. Different flower color.

2. More compact grow habit.

3. Smaller flower size.

Plants of the new cultivar ‘SPCDW1005’ are similar to the pollen parent, Dendrobium ‘181N’ in most horticultural characteristics. However the new variety, ‘SPCDW1005’ differs from ‘181N’ in the following characteristics:

1. Different flower color.

2. Much larger plant size.

3. Much larger flower size.

### COMMERCIAL COMPARISON

‘SPCDW1005’ is similar in most horticultural characteristics to the unpatented commercial variety Dendrobium ‘SPCDW0508’ in most horticultural characteristics. However the new variety, ‘SPCDW1005’ differs from ‘SPCDW0508’ in the following characteristics:

1. Different sepal and pedal color.

2. Glossier leaves.

3. Pedal and sepal shape is rounder.

4. The lateral lobes of the lips are more open.

5. Different lip color.

6. Color pattern shows more netting.

Plants of the new cultivar ‘SPCDW1005’ are also comparable to the unpatented commercial variety Dendrobium ‘SPCDW1013’. The two Dendrobium varieties are similar in most horticultural characteristics; however, the new variety ‘SPCDW1005’ differs in the following ways:

1. Different sepal and pedal color.

2. Different lip color.

3. Pedal and sepal shape is rounder.

### BRIEF DESCRIPTION OF THE PHOTOGRAPH

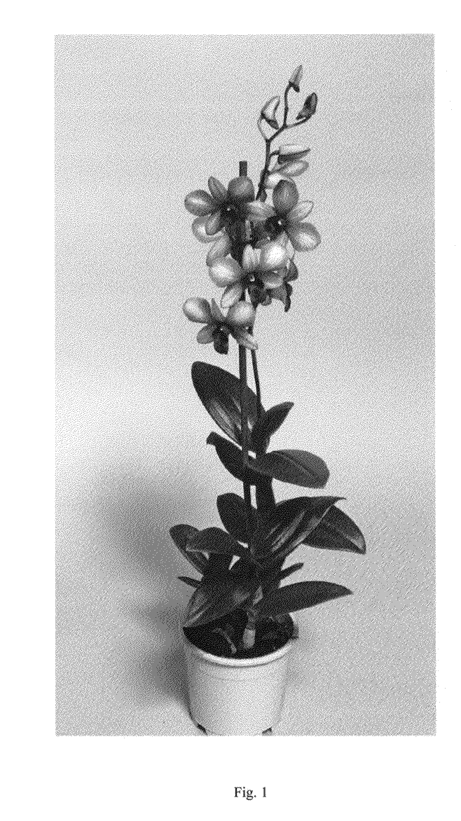

The accompanying photograph in FIG. 1 illustrates in full color a typical plant of ‘SPCDW1005’ grown in a commercial greenhouse in Honselersdijk, the Netherlands. The plant is 1 years old from a rooted cutting, in a 10.5 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007, except where general terms of ordinary dictionary significance are used. The following observations and measurements describe ‘SPCDW1005’ plants grown in a commercial greenhouse in Honselersdijk, the Netherlands. The plant is 1 year old from a rooted cutting, in a 10.5 cm pot. Temperatures ranged between 26° C. and 30° C. day and night. No photoperiodic or chemical treatments were given to the plants. The light sum was set at 5 moles per day and the plant was given shading and artificial light in order to come to the light sum of 5 moles per day. Both shading and artificial lightning were frequently applied in order to achieve the ideal light sum of 5 moles per day. Measurements and numerical values represent averages of typical plant types.
Botanical classification: Dendrobium hybrid. ‘SPCDW1005’
Propagation:
   Type of propagation typically used.—Tissue culture.
   Time to initiate roots.—Tissue culture takes 18 months. After deflasking it takes another 9 to 12 months to get a well-rooted plant under average temperatures ranging between 26 en 30 degrees Celsius. After planting in the end pot, the young plant already has roots and those roots continue to grow in the end pot almost right away and certainly within 8 days.
   Time to produce a rooted cutting/liner.—Tissue culture takes 18months. After deflasking it takes another 9 to 12 months to get a well-rooted plant under average temperatures ranging between 26 en 30 degrees Celsius. After planting in the end pot, the young plant already has roots and those roots continue to grow in the end pot almost right away and certainly within 8 days.
   Root description.—Root Color: White near RHS NN155B Average Diameter: Average: 0.2 cm Average Length: Lateral roots average: 5.2 cm.
Plant:
   Growth habit.—Sympodial, upright.
   Height from top of soil line to top of foliage.—29.4 cm.
   Height from top of soil line to top of flowers.—55.4 cm.
   Plant spread.—22.4 cm.
   Normal pot size.—10.5 cm circular pot.
   Growth rate.—Moderate.
   Number of leaves per plant.—Average: 42.
   Plant stem.—Length: Approximately 12.5 cm. Diameter: Approximately 1.7 cm Aspect: Approximately 75° angle to soil surface (=0°) Strength: Strong Texture: Glabrous, very slightly glossy, axially ribbed Color: Yellow-green, in between near RHS N144A and 152C.
Foliage:
   Leaf.—Arrangement: Distichous Average Length: Approximately 12.0 cm. Average Width: Approximately 5.8 cm. Shape of blade: Ovate to narrow ovate Aspect: Leaves in an average angle of 75° to main stem (=0°) Apex: Acute Base: Sheathing Sheath length: Average 2.7 cm Sheath width: Average 1.6 cm. Sheath color: Yellow-green, near RHS 146D, slightly striped axially, yellow-green; near RHS 147B Margin: Entire Texture of top surface: Glabrous, glossy Texture of bottom surface: Glabrous, moderately glossy Durability to physical stress: High Color: Young foliage upper side: Green to yellow-green; in between near RHS 143A and 144A but darker Young foliage under side: Yellow-green; near RHS 144A Mature foliage upper side: Green; in between near RHS N137A and 143A but darker Mature foliage under side: Yellow-green; near RHS 146A Venation: Type: Parallel Venation coloration: Upper Surface: Green; in between near RHS N137A and 143A but darker Lower Surface: Yellow-green; near RHS 146A.
   Petiole.—No petioles present.
Flowering characteristics:
   Flower bud.—Rate of opening: Approximately 30 days from first sight Length: Approximately 2.4 cm. Diameter: Approximately 1.1 cm. Shape: Ovate, curved Color: Yellow-green; in between near 145A and 145B, margins of unopened sepals and spur red-purple; near RHS 61A.
   Peduncle.—Length: Approximately 29.9 cm. Diameter: Approximately 0.3 cm. Aspect: Approximately 0° angle to stem. Strength: Strong Texture: Glabrous, slightly glossy Color: Yellow-green; near RHS 146C.
   Inflorescence.—Flowers per Stem: Average: 13 Inflorescence Arrangement: Simple raceme Inflorescence Height: Approximately 19.1 cm (from lowest pedicel to top of inflorescence). Inflorescence Width: Approximately 8.5 cm.
   Individual flower.—Flower Type: Single, orchidaceous (zygomorphic), typical for Dendrobium Rate of Opening: After the first flower has opened, it takes approximately four weeks before the top flower opens Shape: orchidaceous (zygomorphic), typical for Dendrobium, with three sepals, two lateral petals and a labellum (third petal). Height: Approximately 4.6 cm. Diameter: Approximately 5.1 cm. Depth: Approximately 2.3 cm. Fragrance: No fragrance Persistent?: Non-persistent Quantity of Petals: Average: 3 (two lateral petals and a labellum (=third petal)) Only the two lateral petals are described here as such.
   Petals.—Arrangement: Oppositely placed on each side of the column Length: Approximately 2.8 cm. Width: Approximately 1.9 cm. Shape: Obovate, slightly curved Apex: Rounded Base: Cuneate Margin: Entire Appearance: Both sides matte, velvety Texture, upper forward surface: glabrous, velvety Texture, lower back surface: glabrous, velvety Color: When opening, forward surface: Yellow-green; in between near RHS 145A to 145B and 146A, veins and margins red-purple; near RHS 71A, base lighter; near RHS 71B When opening, back surface: Yellow-green; RHS 145B, veins and margins purple; near RHS N77B. Fully Opened, forward surface: Yellow-green; RHS 145C, veins and margins red-purple; near RHS 72B, base slightly darker; near RHS 72A. Fully Opened, back surface: Yellow-green; RHS 145C, veins and margins purple; near RHS N78C Fading, forward surface: not fading (colours similar to “Fully Opened”) Fading, back surface: not fading (colours similar to “Fully Opened”) Color Venation Fully Opened, forward surface: As surface (colours similar to “Fully Opened”) Color Venation Fully Opened, back surface: As surface (colours similar to “Fully Opened”).
   Labellum.—Shape: Deltoid, three-lobed, strongly involute Length: Approximately 1.8 cm Width: Approximately 1.6 cm (when “folded open”, approximately 2.6 cm). Texture: glabrous, velvety Appearance: Matte, moderately velvety Tip: Rounded Base: Broad cuneate Color: When opening, forward surface: Purple; near RHS N79B, base lighter; near RHS N79C. When opening, back surface: Central lobe yellow-green; 145D and lighter, tip purple; near RHS N79B, lateral lobes purple; near RHS N79B to N79C. Fully Opened, forward surface: Purple; near RHS N79B, slightly blotched (in between veins) yellow-green; near RHS 146D, base lighter; near RHS N79C. Fully Opened, back surface: Central lobe yellow-green; 145D and lighter, tip and lateral lobes purple; near RHS N79B. Fading, forward surface: (colours similar to “Fully Opened”) Fading, back surface: (colours similar to “Fully Opened”) Color Venation Fully Opened, forward surface: Purple; near RHS N79B Color Venation Fully Opened, back surface: As surface (colours similar to “Fully Opened”) Margin: Entire Callosites: Dimensions: Average: 0.6 cm long and 0.5 cm wide Location: central placed on the labellum, coloured purple; near RHS N79A to N79B.
   Sepals.—Quantity: Average: 3 Appearance: Matte, velvety Length: Approximately 2.8 cm. Width: Dorsal sepal, approximately 1.2 cm. Lateral sepals, approximately 1.4 cm Shape: Narrow ovate, dorsal sepal oblong, slightly curved Apex: Acute Base: Broad cuneate Margin: Entire Texture: glabrous, velvety Color: Forward surface: Yellow-green; near RHS 145B, margins, base and outer tip purple; near RHS N78C, veined darker; near RHS N78B Back surface: Yellow-green; near RHS 145C, margins and base purple; Near RHS N78C.
   Pedicels.—Length: Approximately 3.9 cm. Diameter: Approximately 0.2 cm. Angle: Approximately 60° angle to peduncle (=0°) Strength: Moderately strong Texture: Glabrous, moderately glossy Color: Yellow-green; near RHS 145C, base darker; 145A to 145B.
Reproductive organs: Stamens, style and stigma fused into column? Yes Column Length: Average: 0.6 cm Column Diameter: Average: 0.7 mm Column Color: Purple; near RHS N79B to N79C Ovary Position: Superior, placed directly above stigma Diameter: Average: 0.2 cm Color: Purple; near RHS N79B to N79C Stigma: Shape: Clavate Color: Green-white; near RHS 157A Dimensions: Average: 0.3 cm high and 0.3 cm wide Pollinia: Color: Yellow-orange; near RHS 14A Dimensions: Average: 0.075 cm long and 0.1 cm wide Quantity: 2.
Other characteristics:
   Disease and pest resistance.—High tolerance for typical diseases and pests of Dendrobium.
   Drought tolerance and cold tolerance.—Normal for Genus.
   Fruit/seed production.—No fruits/seeds detected to date.

What is claimed is:

1. A new and distinct cultivar of Dendrobium plant named ‘SPCDW1005’ as herein illustrated and described.
